(19) United States
(12) Patent Application Publication
(10) Pub. No.: US 2009/0326013 A1
(43) Pub. Date: Dec. 31, 2009

(54) ISOMERS OF INOSITOL NIACINATE AND USES THEREOF

(76) Inventor: Curt Hendrix, West Lake Village, CA (US)

Correspondence Address: VENABLE LLP, P.O. BOX 34385, WASHINGTON, DC, 20043-9998, US

(21) Appl. No.: 12/441,628

(22) PCT Filed: Feb. 29, 2008
(86) PCT No.: PCT/US08/02735

§ 371 Date: Mar. 17, 2009

Related U.S. Application Data
(60) Provisional application No. 60/892,456, filed on Mar. 1, 2007.
(60) Provisional application No. 60/960,058, filed on Sep. 13, 2007.

(51) Int. Cl. A61K 31/455

(52) U.S. Cl. 514/356

(57) ABSTRACT

An ester formed from an inositol or an inositol derivative and niacin, wherein the inositol or the inositol derivatives comprises a stereoisomer selected from allo-inositol, cis-inositol, epi-inositol, muco-inositol, neo-inositol, scyllo-inositol, D-chiro-inositol and L-chiro-inositol, or pharmaceutically acceptable salts thereof. Examples of esters include inositol hexaniacinates such as allo-inositol hexaniacinate and cis-inositol hexaniacinate. The esters can be used to treat any disorder that is treatable with niacin therapy such as dyslipidemia, hypercholesterolemia, hyperlipidemia or cardiovascular disease. The esters can be administered with other agents such as HMG-CoA reductase inhibitors, statins, fibrates, activators of peroxisome proliferator activated receptors poli-cosanol, phytosterols, tocotrienols, calcium, bile acid sequestrants, guar gum and free niacin. The invention includes pharmaceutical compositions containing these compounds.

### FIELD OF INVENTION

This invention is a new compounds and methods for it use in the treatment of a broad range of diseases including, but not limited to, hypercholesterolemia, hyperlipidemia and cardiovascular disease. More particularly, the invention is directed to isomers of inositol hexaniacinate and uses thereof.

### BACKGROUND OF THE INVENTION

Various forms of dyslipidemia, including hypercholesterolemia, hyperlipidemia, hypertriglyceridemia, hyperlipoproteinemia, hypocholesterolemia hypolipoproteinemia and imbalances of lipids, lipoproteins and/or triglycerides, as well as and cardiovascular disease are increasingly prevalent in Western industrial societies. The reasons for this are not completely understood, but may relate partly to a genetic predisposition to these conditions and partly to a diet high in saturated fats, together with an increasingly sedentary lifestyle as manual labor becomes increasingly less necessary. Hypercholesterolemia and hyperlipidemia are very significant conditions, because they predispose individuals to cardiovascular disease, including atherosclerosis, myocardial infarction (heart attack), and stroke.

Specific forms of hyperlipidemia include, for example, hypercholesterolemia, familial dysbetalipoproteinemia, diabetic dyslipidemia, nephrotic dyslipidemia and familial combined hyperlipidemia. Hypercholesterolemia is characterized by an elevation in serum low density lipoprotein-cholesterol and serum total cholesterol. Low density lipoprotein (LDL—cholesterol) transports cholesterol in the blood. Familial dysbetalipoproteinemia, also known as Type III hyperlipidemia, is characterized by an accumulation of very low density lipoprotein-cholesterol (VLDL-cholesterol) particles called betaVLDLs in the serum. Also associated with this condition is a replacement of normal apolipoprotein E3 with abnormal isoform apolipoprotein E2. Diabetic dyslipidemia is characterized by multiple lipoprotein abnormalities, such as an overproduction of VLDL-cholesterol, abnormal VLDL triglyceride lipolysis, reduced LDL-cholesterol receptor activity and, on occasion, Type III hyperlipidemia. Nephrotic dyslipidemia, associated with malfunction of the kidneys, is difficult to treat and frequently includes hypercholesterolemia and hypertriglyceridemia. Familial combined hyperlipidemia is characterized by multiple phenotypes of hyperlipidemia, i.e., Type IIa, IIb, IV, V or hyperapobetalipoproteinemia.

It is well known that the likelihood of cardiovascular disease can be decreased if the serum lipids, and in particular LDL-cholesterol, can be reduced. It is also well known that the progression of atherosclerosis can be retarded or the regression of atherosclerosis can be induced if serum lipids can be lowered. In such cases, individuals diagnosed with hyperlipidemia or hypercholesterolemia should consider lipid-lowering therapy to retard the progression or induce the regression of atherosclerosis for purposes of reducing their risk-of cardiovascular disease, and in particular coronary artery disease. Such therapy will reduce the risk of stroke and mycardial infarction, among other consequences. In addition, certain individuals with what are considered normal blood lipid levels can develop cardiovascular disease. In these individuals other factors like lipid peroxidation and high levels of Lp(a) can lead to atherogenesis despite relatively normal cholesterol and lipid levels.

Hypertriglyceridemia is also an independent risk factor for cardiovascular disease, such as coronary artery disease. Many people with hyperlipidemia or hypercholesterolemia also have elevated triglyceride levels. It is known that a reduction in elevated triglycerides can result in the secondary lowering of cholesterol. Hypertriglyceridemic individuals should also consider lipid-lowering therapy to reduce their elevated triglycerides for purposes of decreasing their incidence of atherosclerosis and coronary artery disease. Such therapy is also recommended for individuals who have already experienced an occurrence of stroke or myocardial infarction.

Cholesterol is transported in the blood by lipoprotein complexes, such as VLDL-cholesterol, LDL-cholesterol, and high density lipoprotein-cholesterol (HDL-cholesterol). LDL-cholesterol carries cholesterol in the blood to the subendothelial spaces of blood vessel walls. It is believed that peroxidation of LDL-cholesterol within the subendothelial space of blood vessel walls leads to atherosclerotic plaque formation. HDL-cholesterol, on the other hand, is believed to counter plaque formation and delay or prevent the onset of cardiovascular disease and atherosclerotic symptoms. Several subtypes of HDL-cholesterol, such as HDL₁-cholesterol, HDL₂-cholesterol and HDL₃-cholesterol, have been identified to date.

Numerous methods have been proposed for reducing elevated cholesterol levels and for increasing HDL-cholesterol levels. Typically, these methods include diet modification and/or daily administration of lipid-altering or hypolipidemic agents. Another proposed method is based on periodic plasma delipidation by a continuous flow filtration system, as described in U.S. Pat. No. 4,895,558.

Several types of hypolipidemic agents have been developed to treat individuals with hyperlipidemia or hypercholesterolemia or that have normal lipid profiles but have been diagnosed with cardiovascular disease. In general, these agents act by (1) reducing the production of the serum lipoproteins or lipids, or (2) enhancing removal of lipoproteins or lipids from the serum or plasma. Examples of drugs that lower the concentration of serum lipoproteins or lipids include statins and other inhibitors of HMG-CoA reductase, the rate controlling enzyme in the biosynthetic pathway of cholesterol, and fibrates, which most likely by activating peroxisome proliferator activated receptors (PPARs), particularly PPARα. Exemplary statins include mevastatin, lovastatin, also referred to as mevinolin, pravastatin, lactones of pravastatin, velostatin, also referred to as synvinolin, simvastatin, rivastatin; fluvastatin; atorvastatin; and cerivastatin. Fibrates are generally fibric acid derivatives, such as gemfibrozil, clofibrate, bezafibrate, fenofibrate, ciprofibrate and clinofibrate.

Other drugs that can lower serum cholesterol include, for example, nicotinic acid, bile acid sequestrants, e.g., cholestyramine, colestipol DEA-Sephadex (Secholex® and Polidexide®), probucol and related compounds as disclosed in U.S. Pat. No. 3,674,836, lipostabil (Rhone-Poulenc), Eisai E5050 (an N-substituted ethanolamine derivative), imanixil (HOE-402), tetrahydrolipstatin (THL), isitigmastanyiphosphoryl-choline (SPC, Roche), aminocyclodextrin (Tanabe Seiyoku), Ajirlomoto AJ-814 (azulene derivative), melinamide (Sumitomo), Sandoz 58-035, American Cyanamid CL-277,082 and CL-283,546 (disubstituted urea derivatives), ronicol (which has an alcohol which corresponds to nicotinic acid), neomycin, p-aminosalicylic acid, aspirin, quaternary amine poly(diallyldimethylammonium chloride) and ionenes such as disclosed in U.S. Pat. No. 4,027,009, poly(diallylmethylamine) derivatives such as disclosed in U.S. Pat. No. 4,759,923, omega-3-fatty acids found in various fish oil supplements, and other known serum cholesterol lowering agents such as those described in U.S. Pat. No. 5,200,424; European Patent Application No. 0065835A1, European Patent No. 164-698-A, G.B. Patent No. 1,586,152 and G.B. Patent Application No. 2162-179-A.

HMG-CoA reductase inhibitors such as statins have been used to treat hyperlipidemia. These compounds are known to exhibit beneficial effects of reducing total cholesterol and LDL-cholesterol in the human body and elevating HDL-cholesterol levels in some individuals. Grundy S M, New Eng J. Med., 319 (1):24-32 (1988) at 25-26 and 31. Examples of HMG-CoA reductase inhibitors, generally referred to as statins, include: (1) mevastatin, U.S. Pat. No. 3,983,140; (2) lovastatin, also referred to as mevinolin, U.S. Pat. No. 4,231,938; (3) pravastatin, U.S. Pat. Nos. 4,346,227 and 4,410,629; (4) lactones of pravastatin, U.S. Pat. No. 4,448,979; (5) velostatin, also referred to as synvinolin; (6) simvastatin, U.S. Pat. Nos. 4,448,784 and 4,450,171; (7) rivastatin; (8) fluvastatin; (9) atorvastatin; and (10) cerivastatin. For other examples of HMGCoA reductase inhibitors, see U.S. Pat. Nos. 5,217,992; 5,196,440; 5,189,180; 5,166,364; 5,157,134; 5,110,940; 5,106,992; 5,099,035; 5,081,136; 5,049,696; 5,049,577; 5,025,017; 5,011,947; 5,010,105; 4,970,221; 4,940,800; 4,866,058; 4,686,237; 4,647,576; European Application Nos. 0142146A2 and 0221025A1; and PCT Application Nos. WO 86/03488 and WO 86107054. The conversion of HMG-CoA to mevalonate is an early step in the biosynthesis of cholesterol. Inhibition of HMGCoA reductase, which interferes with the production of mevalonate, is the basis by which the HMG-CoA reductase inhibitors exert their total cholesterol-lowering and LDL-cholesterol-lowering effects. Grundy S M, New Eng. J. Med., 319(1):24-32, at 25 and 26 (Jul. 7, 1988).

However, HMG-CoA reductase inhibitors are not without drawback. They are known to induce hepatotoxicity, myopathy and rhabdomyolysis, as reported in, for example, Garnett W R, Am. J. Cardiol., 78 (Suppl 6A):20-25 (1996), The Lovastatin Pravastatin Study Group:, Am. J. Cardiol., 71:810-815 (1993), Dujovne C A et al., Am. J. Med., 91 (Suppl 1 B):25S-30S (1991); and Mantell G M et al., Am. J. Cardiol., 66:11 B-1 5B (1990). Statins do not significantly reduce triglycerides and result in minimal increase of HDL. Additionally they have little impact on Lp(a) and may even increase it.

The Physicians' Desk Reference (PDR) 50th Ed., page 1700, column 3 (1996), reports that lovastatin should be used with caution in patients who have a past history of liver disease, and that lovastatin therapy is contraindicated for those individuals with active liver disease or unexplained persistent elevations of serum transaminases. The 1996 PDR further reports (page 1701, column 1) that rhabdomyolysis has been associated with lovastatin therapy alone and when combined with lipid-lowering doses (about 1 g/day) of nicotinic acid, and that physicians contemplating combined therapy with lovastatin and lipid-lowering doses of nicotinic acid should carefully weigh the potential benefits and risks and should carefully monitor individuals for any signs and symptoms of muscle pain, tenderness, or weakness, particularly during the initial months of therapy and during any periods of upward dosage titration of either drug.

### SUMMARY OF THE INVENTION

Because of the deficiencies and side effects of current treatment modalities, there is a need for improved compounds, compositions and methods that can be used to treat hyperlipidemia, hypercholesterolemia and hypertriglyceridemia, or can be used to lower blood lipid levels, blood cholesterol levels, or blood triglyceride levels in patients with normal levels of these physiological parameters who are at risk of cardiovascular disease or who have already experienced an episode of cardiovascular disease. There is further a need for improved compositions and methods that reduce other cardiovascular risk factors like lipid peroxidation, and levels of Lp(a) and avoid the side effects such as flushing associated with the administration of niacin and that also avoid the risks of liver and muscle damage associated with the statins and other anti-lipidemic drugs. There is further a need for improved compositions that reverse cardiovascular plaques as well as improved compositions that provide protection for an extended period of time without complex dosing regimens. Furthermore, there is a need for improved compositions and methods that are particularly beneficial to individuals at risk for cardiovascular disease because of existing diabetic symptoms or metabolic syndrome, and can be used to treat cardiovascular disease.

Esters of niacin with inositol stereoisomers other than myo-inositol can have physical chemical and physiological or pharmacokinetic properties that are surprisingly different than myo-insoitol hexaniacinate. In particular, at least allo-inositol hexaniacinate is a newly generated niacin inositol ester that can be used in the treatment of cardiovascular disease, hypercholesterolemia and hyperlipidemia, as well as other dissorders that can be treated with niacin. Its different properties can be expected to result in delivery of niacin that is more easily controlled, and the flushing or burning sensation associated with niacin treatment can be eliminated or considerably reduced to a level which is more acceptable to patients. The inventive isomers of IHN can be used in all instances where niacin in its various forms have been used in the past. Other isomers of inositol can be reacted to form niacinates which can have unique physical chemical properties and provide unexpected physiological benefits for a variety of indications that are amenable to treatment with niacin, and can be superior to the physcial chemical, physiological and or pharmacokinetic properties of myo-inositol hexaniacinate.

The invention is an ester formed from an inositol or an inositol derivative and niacin, wherein the inositol is, or the inositol derivatives is from, comprises a stereoisomer selected from allo-inositol, cis-inositol, epi-inositol, muco-inositol, neo-inositol, scyllo-inositol, D-chiro-inositol and L-chiro-inositol. The invention includes pharmaceutically acceptable salts of the esters. The esters can be inositol hexaniacinates, such as allo-inositol hexaniacinate and cis-inositol hexaniacinate. The invention is also a composition, for example a pharmaceutical composition, comprising an ester of the invention. The composition can also include a second pharmaceutically active moiety, for example, niacin, HMG-CoA reductase inhibitors, statins, fibrates, activators of peroxisome proliferator activated receptors policosanol, phytosterols, tocotrienols, calcium, bile acid sequestrants, and guar gum.

The invention is also a method of treating a disorder treatable with niacin comprising delivering a therapeutically effective amount of a composition that includes an ester as described above and, optionally, a second pharmaceutically active moiety. Disorders treatable with niacin include dyslipodemia, hypercholesterolemia, hyperlipidemia, hypertriglyceridemia, hyperlipoproteinemia, hypocholesterolemia hypolipoproteinemia and imbalances of lipids, lipoproteins and/or triglycerides; cardiovascuolar disease; diabetes or inuslin resistance; peripheral vascular diseases including Raynaud's disease, thrombotic risk, intermittent claudication, hypertension, vascular insufficiency and restless leg syndrome and other peripheral artery disease, dysmennorhea, carcinogenesis, anxiety depression, PMS, and treatment of metabolic syndrome due to insulin resistance. The composition can be delivered orally.

### DESCRIPTION OF DRAWINGS

FIG. 1 shows the main confirmations of scyllo-inositol.

FIG. 2 shows the main confirmations of myo-inositol.

FIG. 3 shows the main confirmations of cis-inositol.

FIG. 4 shows the main confirmations of allo-inositol.

FIG. 5 is a graphical representation of calculated dipole moments for the minimum energy confirmations of the IHN compounds listed in Table 2.

FIG. 6 is a graphical representation of calculated steric energy for the minimum energy confirmations of the IHN compounds listed in Table 2.

FIG. 7 is a graph comparing the hydrolysis of myo-inositol hexaniacinate and scyllo-inositol hexaniacinate showing the release of niacin in SGF at a pH of 1.1.

FIG. 8 is a graph comparing the hydrolysis of allo-IHN with myo-IHN in a SGF solution at pH 1.1 showing the release of niacin.

FIG. 9 is a graph comparing the hydrolysis of allo-IHN with myo-IHN showing the release of niacin in a SIF solution at pH 7.4.

### DETAILED DESCRIPTION

Embodiments of the invention are discussed in detail below. In describing embodiments, specific terminology is employed for the sake of clarity. However, the invention is not intended to be limited to the specific terminology so selected. While specific exemplary embodiments are discussed, it should be understood that this is done for illustration purposes only. A person skilled in the relevant art will recognize that other components and configurations can be used without parting from the spirit and scope of the invention. All references cited herein are incorporated by reference as if each had been individually incorporated.

The terms “niacin” and “nicotinic acid” are used interchangeably herein to refer to pyridine-3-carboxylic acid. The terms “niacinate” and “nicotinate” are used to refer to esters of niacin formed by reaction of a hydroxyl containing compound with pyridine-3-carboxylic acid. In the absence of a designation as to the number of niacin moieties, the term niacinate refers to an ester having unspecified number of niacin moieties, for example, mono-esters, di-esters, tri-esters, tetra-esters, penta-esters, hexa-esters, etc., as well as mixtures thereof.

The term “inositol” is used herein to describe the free sugar, 1,2,3,4,5,6-cyclohexanehexaol. As will be appreciated, the term inositol as used in the literature frequently refers to the myo-isomer or cis-1,2,3,5-trans-4,6-Cyclohexanehexyl.

As used herein. “IHN” refers to inositol hexaniacinate. Unless preceded by a prefix designating the stereoisomer of inositol, “IHN” shall be taken to mean an inositol hexaniacinate prepared from inositol of unspecified stereochemistry, inositols of mixed stereochemistry or myo-inositol. IHN prepared from specific isomers of inositol are indicated by attaching the prefix designating the inositol isomer prior to “IHN.” For example, the hexaester of niacin and allo-inositol is referred to as allo-IHN.

As used herein, “inositol derivative” refers to a compound that includes an inositol moiety having one or more functionalized hydroxyl groups, but retaining one or more free hydroxyl groups. Inositol derivatives may have hydroxyl groups functionalized to be, for example, an ether or an ester. Examples of inositol derivatives include the methyl ethers D-Pinitol and L-Quebrachitol, and inositol phosphates and phosphonates that are esterified with one to five phosphate or phosphonate groups. The phosphate and phosphonate groups may include the substitution of sulfur for one or more oxygen atoms to form thio-ester or thio-alkyl groups.

An exemplary application for compounds that generate niacin in the body is the provision of lipid lowering or other cardiovascular benefits. In this disclosure, this benefit may at times be the only indication or benefit described with respect to a particular compound or treatment regime. However, as will be appreciated by persons skilled in the art, any condition treatable with niacin can be subjected to treatment with the compounds and to the treatment regimes described herein. Thus, reference to a single treatment or a single benefit of niacin therapy is intended to be exemplary and not a limitation on the use of the compounds or the treatment regimen specifically identified herein.

As used herein, “treat” or “treatment” refers to the causation of any detectable improvement of a disorder or condition that is clinically significant and does not require or demand a cure.

Free inositol is part of the vitamin B-group (referred to as vitamin Bh). Inositol can exist as a number of steroisomers, illustrated in Scheme 1. Naturally occurring isomers of inositol are the myo-, scyllo-, muco-, neo-, D-chiro, and L-chiro forms. Additional isomers of inositol which can be produced synthetically are the epi-, cis- and allo-forms. It is important to distinguish inositol, the free sugar, from IHN. There are examples in the literature of IHN being referred to as inositol.

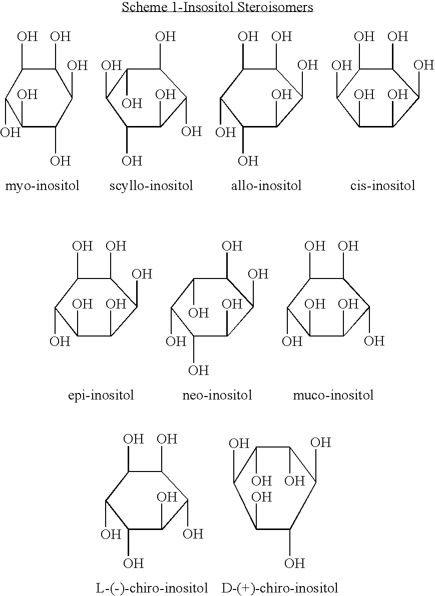

Various naturally occurring inositol derivatives, such as 4-O-methyl-D-myo-inositol, 1,3-di-O-methyl-D-myo-inositol, D-Pinitol and L-Quebrachitol are found in a wide variety of plants. D-Pinitol can be isolated from sugar pines and L-Quebrachitol is obtained from rubber trees, and are based on D-chiro-inositol and L-chiro-inositol, respectively. Both D-Pinitol and L-Quebrachitol are readily available in large quantities and serve as versatile starting materials in synthetic organic chemistry. Inositol and its derivatives play an important role in animal and human metabolism. The human body is able to produce free inositol and the regulation of its level is of therapeutic relevance. An example of important inositol derivatives in mammals are phosphatidylinositols. They often constitute a component of lecithin and can act as lipotropic agents, helping to emulsify fats. Furthermore, phosphatidylinositols play a key role in signal transduction in cells.

Myo-inositol triphosphate (IP-3), formed from membranous bound phosphatidylinositol, acts as a second messenger and is important in the control of many cellular processes because it regulates internal calcium signals. The phosphatidylinositol pathways are of major importance in the context of physiological processes and disease conditions including arthritis, pain, inflammation, platelet aggregation, and, possibly, oncogenesis.

It has been recommended that diabetic patients take extra free inositol. Even though the body is able to produce its own inositol from glucose, administration of inositol shows some success in improving the nerve function in diabetic patients who have experienced pain and numbness due to nerve degeneration.

Some problems that are considered to be associated with low levels of inositol in the body are eczema, constipation, eye problems, hair loss, and elevation of cholesterol.

Niacin, also referred to as nicotinic acid or vitamin B3, is vital to cellular metabolism and has gained attention in the treatment of various diseases including several cardiovascular conditions. For example, niacin has been used in the treatment of hyperlipidemia or hypercholesterolemia. This compound has long been known to exhibit the beneficial effects of reducing total cholesterol, VLDL-cholesterol and VLDL-cholesterol remnants, LDL-cholesterol triglycerides, and Lp(a), in the human body, while increasing desirable HDL-cholesterol.

For therapeutic purposes, niacin is normally administered three times per day after meals. This dosing regimen is known to provide a very beneficial effect on blood lipids as discussed in Knopp et al., “Contrasting Effects of Unmodified and Time-Release Forms of Niacin on Lipoproteins in Hyperlipidemic Subjects: Clues to Mechanism of Action of Niacin”; Metabolism, 34(7): 642-647 (1985). The chief advantage of this profile is the ability of niacin to decrease total cholesterol, LDL-cholesterol, triglycerides and Lp(a) while increasing HDL-cholesterol particles. While such a regimen produces beneficial effects, cutaneous flushing and a burning sensation over the skin surfaces often occur in the individuals to whom the niacin is administered. While these side effects are uncomfortable, they do not present a danger to the patient. However, many patients will cease niacin therapy because of these side effects.

In order to avoid or reduce the cutaneous flushing and other unpleasant side effects resulting from niacin therapy, a number of agents have been suggested for administration with an effective antihyperlipidemic amount of niacin, such as guar gum as reported in U.S. Pat. No. 4,965,252, mineral salts as disclosed in U.S. Pat. No. 5,023,245, inorganic magnesium salts as reported in U.S. Pat. No. 4,911,917, and non-steroidal anti-inflammatories, such as aspirin, as disclosed in PCT Application No. 96/32942. These agents have been reported to avoid or reduce the cutaneous flushing side effect commonly associated with niacin divided dose treatment.

Another method of avoiding or reducing the side effects associated with immediate release niacin is the use of extended or sustained release formulations. Extended or sustained release formulations are designed to slowly release the active ingredient from the tablet or capsule, which allows a reduction in dosing frequency as compared to the typical dosing frequency associated with conventional or immediate dosage forms. The slow drug release reduces and prolongs blood levels of the drug and, thus, minimizes or lessens the cutaneous flushing side effects that are associated with conventional or immediate release niacin products. Sustained release formulations of niacin have been developed, such as Nicobid® capsules (Rhone-Poulenc Rorer), Endur-acin® (Innovite Corporation), and the formulations described in U.S. Pat. Nos. 5,126,145 and 5,268,181, which describe a sustained release niacin formulation containing two different types of hydroxypropylmethylcelluloses and a hydrophobic component.

Studies in hyperlipidemic patients have been conducted with a number of sustained release niacin products. While initial studies indicated a performance similar to immediate release niacin, other studies have demonstrated that the sustained release products do not have the same advantageous lipid-altering effects as immediate release niacin. The major disadvantage of the sustained release formulations, as reported in Knopp et al.: Metabolism, 34(7): 642-647 (1985), is 1) a significantly lower reduction in triglycerides (−2% for the sustained release versus −38% for the immediate release) and 2) lower increase in HDL-cholesterol (+8% for the sustained release versus +22% for the immediate release) and HDL₂-cholesterol particles, which are known by the art to be most beneficial (−5% for the sustained release versus +37% for the immediate release).

Additionally, sustained release niacin formulations are known to cause greater incidences of liver toxicity, as described in Henken et al., Am. J. Med., 91: 1991 (1991) and Dalton et al., Am. J. Med., 93: 102 (1992). There is also great concern regarding the potential of these formulations in disrupting glucose metabolism and uric acid levels.

“A Comparison of the Efficacy and Toxic Effects of Sustained- vs. Immediate-Release Niacin in Hypercholesterolemic Patients”, McKenney et al., J. Am. Med. Assoc., 271(9): 672 (1994) presented the results of a study of twenty-three patients regarding liver toxicity problems associated with a sustained release form of niacin. Eighteen patients (78%) were forced to withdraw because of changes seen in liver function tests (LFTs) indicating potential liver damage. The conclusion of the authors of that article was that the sustained release form of niacin “should be restricted from use.” Similar conclusions have been reached by other health care professionals, including information presented in an article by representatives of the Food and Drug Administration entitled “Hepatic Toxicity of Unmodified and Time-Release Preparations of Niacin”, Rader et al., Am. J. Med., 92:77 (January, 1992).

Of particular interest is the use of niacin to treat hyperlipidemias and other dyslipodemias, and peripheral vascular disorders such as Raynaud's disease and other peripheral artery diseases and intermittent claudication. In some cases there appears to be a correlation between peripheral artery disease and cardiovascular disease. IHN has been used as a treatment of peripheral artery disease. However, while niacin has numerous therapeutic benefits, it also presents some unacceptable side effects, such as flushing and a burning sensation, which many patients refuse to tolerate.

Besides cardiovascular applications, there are also a number of other conditions which respond favorably to niacin therapy. For example, elevated levels of acetaldehyde are postulated to contribute to addiction in alcoholics while a possible deficiency of NAD is believed to cause restlessness and irritability in this population. Niacin oxidizes alcohol to reduce acetaldehyde levels and also saturates NAD receptors in the brain to abolish a possible deficiency of NAD. A five year study of 507 alcoholics receiving three (3) or more grams of niacin daily reported that 30-60% of alcoholics benefit from supplementation by reduced recidivism and symptom reduction. Most studies examined recommended a minimum of 500 mg daily for therapeutic efficacy. A concern lies with the supplementation of high doses of niacin to a population with already compromised liver status.

Grundy S M, New Eng. J. Med., 319 (1):24-33 (1988), reported that HMG-CoA reductase inhibitors when used alone (pages 29-30) and niacin when used alone (page 24) are effective in reducing elevated cholesterol plasma levels. Grundy further reports that “[b]ecause of their efficacy . . . bile acid sequestrants (cholestyramine and colestipol) and niacin are probably the drugs of first choice for hypercholesterolemia. Although these drugs can be highly effective and are satisfactory for use in many patients with high cholesterol levels, they unfortunately are not well tolerated by all patients. Therefore, in spite of their proved usefulness, bile acid sequestrants and niacin are not ideal cholesterol-lowering agents” (page 24, column 2, lines 10-25). Still further, Grundy reports that the “ . . . administration of [HMG-CoA] reductase inhibitors twice a day is somewhat more effective than administration once a day, at the same total dosage” (page 30, column 1, lines 13-17). Grundy also reports “ . . . that the combination of lovastatin and cyclosporine, gemfibrozil or niacin may predispose patients to myopathy and occasionally even to rhabdomyolysis” (page 29, column 1, lines 7-11). Still further, that “the combination of lovastatin and niacin has not been shown to be safe in a controlled clinical trial; furthermore, a manifestation of an adverse interaction between the agents, such as myopathy, could occur” (page 30, column 1, lines 54-59). Gardner S F et al., Pharmacotherapy, 16 (3):421-423 (1996); Pastemak R C et al., Ann Intern. Med., 125 (7):529-540 (1996), O'Keefe J H et al., Am. J. Cardiol., 76:480-484 (1995), and Davignon J et al., Am. J. Cardiol., 73:339-345 (1994) also address these issues.

In Vacek J L et al., Am. J. Cardiol., 76:182-184 (1995), it is reported that “ . . . because of the present state of knowledge of the risks of hepatotoxicity with slow-release forms of niacin, this form of the drug should probably not be used [in combination with lovastatin] in future trials or clinical practice.” This is consistent with the 1996 PDR which reports (page 1701, column 1) that cases of myopathy have been associated with patients taking lovastatin concomitantly with lipid-lowering doses of niacin. Similar contraindications are indicated for (1) fluvastatin (1996 PDR, page 2267—column 3, page 2268, column 1), (2) pravastatin (1996 PDR, page 767, column 1), and (3) simvastatin (PDR, page 1777, column 2). Still further, the 1996 PDR states that concomitant therapy with HMG-CoA reductase inhibitors and lipid lowering doses of niacin is generally not recommended (1996 PDR, page 768, column 3). It is therefore concluded that these agents have the potential for causing serious side effects, particularly in individuals who have liver problems or other problems that can predispose them to these side effects.

Consistent with the reports by Vacek J L et al. and the 1996 PDR, an article by Jacobson T A and Amorosa L F, Am. J. Cardiol., 73:25 D-29D (1994), reports that because “[a]bnormalities in liver enzyme profiles and fulminant hepatic failure have also been associated with the use of niacin, particularly sustained-release preparations . . . the use of fluvastatin in combination with a sustained release niacin preparation cannot generally be recommended based upon this study, which only examined crystalline or immediate release niacin.”

Current products on the market for delivery of niacin can be classified as immediate release, extended release or slow release forms. Immediate release compositions contain from about 25 mg to about 3,000 mg of niacin. The niacin reaches the blood stream in about 0.5 hours and is all released in about 2.5 hours. Extended release compositions, such as Niaspan™, contain from about 100 mg to about 3,000 mg of niacin. About 6-20% of the niacins from this extended release product is released into the blood stream 0.5-2.5 hours following ingestion with about 75% of the niacin is released by about 5-9 hours following ingestion, with a Tₘₐₓ, of 5.6 to 6 hours (U.S. Pat. No. 6,818,229). Further, as is set forth in U.S. Pat. No. 6,080,428, Niaspan™ is to be taken once per day in the evening or at night (i.e., “once per day before going to bed”). Slow release forms contain about the same amount of niacin as immediate release and extended release products. However, the slow release products do not begin to show up in the blood stream until 10 hours following ingestion and continue to be released until about 24 hours from ingestion.

Immediate release and extended release niacin formulations have similar efficacy in reducing blood lipids; however, the effect of the extended release formulation is delayed for several hours. The extended release formulation is promoted as resulting in less flushing than the immediate release form. Also, the slow release formulations are less efficient at reducing blood lipids and have a tendency to increase liver enzymes. However, they have a reduced incidence of flushing when compared to immediate and extended release formulations. The use of nicacin and niacin derivatives to treat dysregulation of lipid metabolism has also been described in G. Domer & F. W. Fischer, “Zur Beeinflussung der Serumlipide and -lipoproteins durch den Hexanicotinsaureester des m-Inositol,” Arzneim. Forsch. 11: 110-113 (1961); A.M.A. El-Enein et al., “The Role of Nicotinic Acid and Inositol Hexaniacinate as Anticholesterolemic and Antilipemic Agents,” Nutrition Rep. Int'l. 28: 899-911 (1983); V. Hutt et al., “Zur Wirkung einer Clofibrat-Inositolnicotinat-Kombination auf Lipide and Lipoproteine bei primarer Hyperlipoproteinamie der Typen IIa, IV and V,” Arzneim. Forsch. 33: 776-779 (1983); W. Kruse et al., “Nocturnal Inhibition of Lipolysis in Man by Nicotinic Acid and Derivatives,” Eur. J. Clin. Pharmacol. 16: 11-15 (1979); and J. G. Wechsler et al., “Lipids and Lipoproteins in Hyperlipidemia Type IIa During Treatment with Different Lipid Lowering Drugs,” Artery 8: 519-529 (1980). Studies have shown that phosphatidylinositol can stimulate reverse cholesterol transport by enhancing the flux of cholesterol into HDL-cholesterol and by promoting the transport of HDL-cholesterol to the liver and bile.

The use of niacin in diabetics is somewhat controversial. Niacin is part of glucose tolerance factor (GTF). Therefore, a deficiency of niacin will interfere with GTF synthesis. Animal studies also indicate that niacin may retard the development of diabetic nephropathy. However, niacin, at least in large doses, may impair glucose tolerance. It is not known whether niacin increases blood glucose by decreasing insulin secretion or by promoting insulin resistance. If niacin increases blood glucose by promoting insulin resistance, then niacin treatment would not be an issue for Type I diabetics since they have virtually no endogenous insulin secretion anyway. A 1977 study combining IHN, most likely myo-IHN, at a dose of 250 mg 3 times daily with Mg-chlorophenoxyisobutyrate for the treatment of hyperlipidemia found no influence on glucose tolerance with this regime. The inositol fraction of the IHN may be beneficial to diabetics as sorbitol accumulation, implicated in many of the long term effects of diabetes, may be a result of inositol loss. Positive studies of inositol for the treatment of diabetic neuropathy have also been reported. A typical therapeutic dose for inositol for the treatment of diabetic neuropathy is in the range of 1 gram or more daily. Because a 600 mg dosage of IHN contains only 90 mg inositol, addition inositol might be required to achieve optimal results.

There is also evidence that niacin may be beneficial for the treatment of dysmennorhea. Hudgins reported on a group of 80 women suffering from painful menstrual cramps who were supplemented with 100 mg niacin twice daily, beginning 7-10 days before the onset of menses and then every 2-3 hours during heavy cramps. Ninety percent of participants experienced significant relief. Therefore niacin releasing agents may be viable as a treatment for dysmennorhea. However, the dosage required during heavy cramping is high enough to cause the unpleasant side effects associated with niacin treatments.

Jacobson et al have initiated studies to evaluate the potential of niacin in the prevention of human carcinogenesis. The known role of ADP-ribose polymer metabolism in limiting carcinogenesis and the dependence of this metabolic function on intracellular NAD levels leads to the prediction that niacin deficiency may enhance carcinogenesis. It therefore appears appropriate to provide niacin supplementation in a safe, well-tolerated form.

Megadoses of niacin have been suggested for treating schizophrenia. Such treatments are controversial as both positive and negative double-blind studies have appeared in the scientific literature. The consensus of many academicians is that niacin therapy is ineffective while others indicate that niacin is primarily effective for early and acute schizophrenics but is ineffective, especially when given alone, for the chronic schizophrenics. The effect of high-dosage niacin supplementation on the liver must also be considered in treating schizophrenia.

Patients with sub-clinical pellagra may develop perceptual changes and neurasthenia and therefore could be mistakenly labeled as schizophrenic but could also benefit from treatment with niacin. Blood niacin levels would help to identify such cases. Other patients who present with schizophrenic syndromes could also benefit from niacin therapy.

While niacin itself has been found to reduce triglyceride and low density lipoprotein (LDL) levels and raise high density lipoprotein (HDL) levels, the degree in which this drug works varies from patient to patient. Niacin may significantly reduce triglycerides and LDL levels in one patient, but may be ineffective in another patient. The mechanism by which niacin works is not completely understood. Further, since the majority of niacin is consumed in the liver by liver enzymes and does not reach the blood stream, abnormal liver function tests, high blood sugar levels and muscle pains may result.

In addition to the conditions mentioned above, niacin has also been implicated as a viable therapy of treatment of hyperthyroidism, multiple sclerosis and tardive dyskinesia. There may be other conditions that could benefit from niacin therapy if an effective niacin releasing agent that would improve patient compliance were available.

Due to side effects described above, a need remains for safer, better tolerated, and perhaps even more effective forms for administering niacin. A compound which has been used as an alternative niacin source is the hexaester of inositol and niacin, referred to as inositol hexaniacinate, inositol hexanicotinate or inositol nicotinate, which will be referred to herein as inositol hexaniacinate or IHN. It should be noted that the published literature sometimes mistakenly refers to IHN as inositol, and the distinction should be taken from the context of any particular report. IHN has been reported to have an apparent lack of the side effects that have been observed with other niacin generating compounds. For some applications, the well-known lipotropic effects of inositol add to the attractiveness of using this compound for the control of dyslipidemia.

Although the term IHN is frequently used rather generically, commercial IHN and IHN as referred to in the literature is most often prepared from myo-inositol to produce myo-inositol hexaniacinate, or myo-IHN. Published literature has addressed the use of myo-IHN for several medical conditions and several references specifically identify commercially available IHN as myo-inositol hexaniacinate. Myo-IHN has a fairly broad range of therapeutic applications. The most well researched conditions include the hyperlipidemias, Raynaud's disease and intermittent claudication. Promising applications which bear further investigation include its use as an alternative to niacin for treatment of stasis ulcers, dysmenorrhea, dermatitis herpetiformis, alcoholism, diabetes, hyperthyroidism, multiple sclerosis, tardive dyskinesia, cancer prevention, peripheral artery disease and hypertension and other conditions amenable to niacin therapy.

IHN consists of six niacin moieties linked to the six hydroxyl groups of the inositol ring. IHN is slowly metabolized in the body, as shown in Scheme 2, into its components, niacin and inositol, with all or substantially all of the niacin groups eventually being cleaved, typically through the loss of individual niacin groups in a step-wise manner. This metabolic cleavage results in a sustained increase level of free niacin in the blood and plasma. Therefore, by administering inositol hexaniacinate the undesired side effects of niacin can be reduced while maintaining its beneficial impact during the treatment of various diseases.

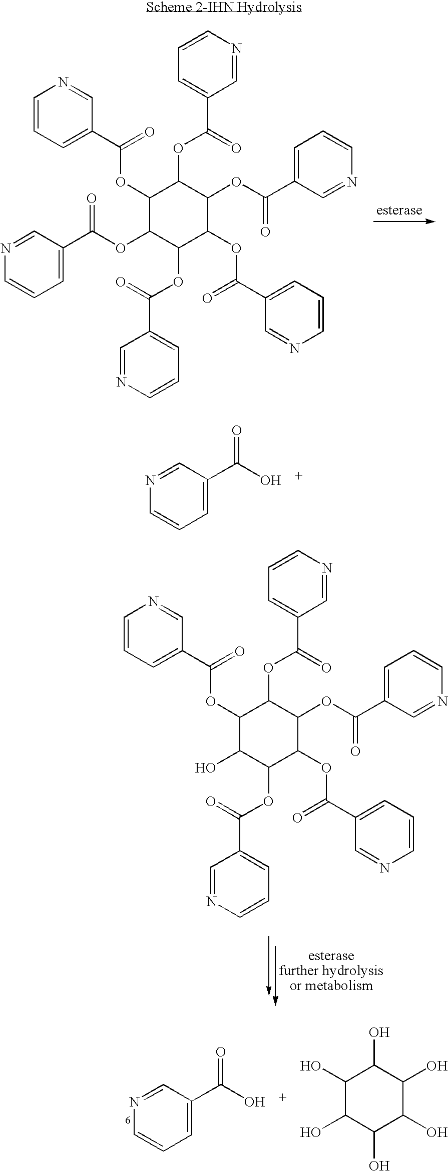

As mentioned above, however, inositol can exist as eight other stereoisomers. Although the simple hydrolysis reaction required to generate niacin from IHN would be expected to proceed comparably for all stereoisomers of inositol, it has surprisingly been found that this is not true. Different steroisomers of IHN have surprisingly different physical chemical properties that can result in differences in physiology and pharmacokinetics. These varied properties can affect the bio-availability of niacin and pharmacokinetics of niacin release. In comparison with myo-IHN, the different physical chemical and physiological properties of other IHN stereoisomers may make one or more of the other inositol niacinate isomers more attractive for a particular application. The properties of allo-IHN appear to make it well suited for a broad range of therapies involving niacin, although the other isomers can have advantages as well.

As will be appreciated, the benefits of the various and specific stereoisomers of IHN can extend to related compounds. For example, other inisitol niacinates such as lower esters, i.e. mono-, di-, tri-, tetra- and penta-niacinates, may be suitable for similar uses or for therapies not yet described. The different niacinates can potentially release niacin at different rates, leading to selecting an IHN based on the rate of niacin release. Similarly, inositol niacinates prepared from inositol derivatives, for example ethers, esters, phosphates and phosphonates, can also have varied release rates and find uses in the applications described herein, as well as other applications.

Scyllo-inositol hexaniacinate (scyllo-IHN), with alternating up and down ester groups, has significantly reduced steric hindrance as compared to other isomers and would be expected to readily and rapidly release a first niacin group once exposed to plasma esterase, and its physiological effect could be expected to be more controllable and more predictable.

The present invention is directed to compounds that are esters of niacin with inositol or inositol derivatives wherein the inositol or inositol derivative comprises a stereoisomer of inositol other than myo-inositol. Steroisomers of inositol other than myo-inositol include allo-inositol, cis-inositol, epi-inositol, muco-inositol, neo-inositol, scyllo-inositol, D-chiro-inositol and L-chiro-inositol. An inositol derivative comprises a steroisomer of inositol other than myo-inositol if the inositol backbone of the inositol derivative is not myo-inositol. Suitable stereoisomers of inositol that may comprise the inositol backbone include allo-inositol, cis-inositol, epi-inositol, mucoinositol, neo-inositol, scyllo-inositol, D-chiro-inositol and L-chiro-inositol.

Esters according to the invention can be formulated into pharmaceutically active compositions by combining the compound with one or more pharmaceutically acceptable excipients. While oral administration is the most commonly intended method, other methods of administration as set forth herein may be appropriate for particular treatment regimens.

Esters according to the invention may be administered for the treatment of disorders and conditions that are amenable to treatment with niacin. Examples of such disorders include dyslipodemia, including hypercholesterolemia, hyperlipidemia, hypertriglyceridemia, hyperlipoproteinemia, hypocholesterolemia hypolipoproteinemia and imbalances of lipids, lipoproteins and/or triglycerides; cardiovascuolar disease; diabetes or inuslin resistance; peripheral vascular diseases including Raynaud's disease, thrombotic risk, intermittent claudication, hypertension, vascular insufficiency and restless leg syndrome and other peripheral artery disease; dysmennorhea; carcinogenesis; anxiety; depression; PMS; and treatment of metabolic syndrome due to insulin resistance. Compositions according to the invention can also be beneficial in reducing fibrinogen and increasing blood viscosity, reducing or alleviating migrane headaches and treating alcoholism and skin diseases such as pruritis and sceleroderma.

Pharmaceutical compositions according to the invention can include additional pharmaceutical moieties that are useful in the treatment of the particular disorder or condition that is targeted. For example, in the case of dyslipidemia and cardiovascular disease, additional pharmaceutical agents can include statins and other inhibitors of HMG-CoA reductase and fibrates, or other activators of PPARs, particularly PPARα. Exemplary statins include mevastatin, lovastatin, also referred to as mevinolin, pravastatin, lactones of pravastatin, velostatin, also referred to as synvinolin, simvastatin, rivastatin; fluvastatin; atorvastatin; and cerivastatin. Fibrates are generally fibric acid derivatives, such as gemfibrozil, clofibrate, bezafibrate, fenofibrate, ciprofibrate and clinofibrate. Other ingredients known to have a beneficial effect on serum lipids and to lower cholesterol, such as, but not limited to policosanol, phytosterols, tocotrienols, calcium, bile acid sequestrants, and guar gum can be added to the composition or co-administered with the inositol niacinate. If present, these ingredients can be added in a therapeutically effective quantity. In some embodiments, the amount of IHN and the additional pharmaceutical ingredient are each present in an amount that is less than the amount of each required to obtain the same effect individually. In this manner, the side effects of each individual ingredient can be reduced or eliminated. Some combinations of IHN and other pharmaceutically active compounds may provide synergistic effects. In addition ingredients such as, for example, L-lysine, L-proline, vitamin C, vitamin E, or other antioxidants that prevent lipid peroxidation, as well as fish oils, phosphatidyl inositols, and pantethines can be added to the composition. If present, these ingredients can also be added in a therapeutically effective quantity.

Pharmaceutical formulations according to the invention comprise an ester of one or more stereoisomers of inositol or inositol derivatives with niacin, or a pharmaceutically acceptable salt thereof, as an active ingredient together with one or more pharmaceutically acceptable carriers, excipients or diluents. Any conventional technique may be used for the preparation of pharmaceutical formulations according to the invention. The active ingredient may be contained in a formulation that provides quick release, sustained release or delayed release after administration to the patient.

Pharmaceutical compositions that are useful in the methods of the invention may be prepared, packaged, or sold in formulations suitable for oral, parenteral and topical administration. Other contemplated formulations include nanoparticles, liposomal preparations, resealed erythrocytes containing the active ingredient, and immunologically-based formulations.

The formulations of the pharmaceutical compositions described herein may be prepared by any method known or hereafter developed. In general, preparation includes bringing the active ingredient into association with a carrier or one or more other additional components, and then, if necessary or desirable, shaping or packaging the product into a desired single- or multi-dose unit.

As used herein, “additional components” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; pharmaceutically acceptable polymeric or hydrophobic materials as well as other components.

Although the descriptions of pharmaceutical compositions provided herein are principally directed to pharmaceutical compositions which are suitable for administration to humans, it will be understood by the skilled artisan, based on this disclosure, that such compositions are generally suitable for administration to any mammal or other animal. Preparation of Compositions Suitable for Administration to Various Animals is Well Understood, and the ordinarily skilled veterinary pharmacologist can design and perform such modifications with routine experimentation based on pharmaceutical compositions for administration to humans.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient in each unit dose is generally equal to the total amount of the active ingredient which would be administered or a convenient fraction of a total dosage amount such as, for example, one-half or one-third of such a dosage.

A formulation of a pharmaceutical composition of the invention suitable for oral administration may in the form of a discrete solid dosage unit. Solid dosage units include, for example, a tablet, a caplet, a hard or soft capsule, a cachet, a troche, or a lozenge. Each solid dosage unit contains a predetermined amount of the active ingredient, for example a unit dose or fraction thereof. Other formulations suitable for administration include, but are not limited to, a powdered or granular formulation, an aqueous or oily suspension, an aqueous or oily solution, or an emulsion. As used herein, an “oily” liquid is one which comprises a carbon or silicon based liquid that is less polar than water.

A tablet comprising the active ingredient may be made, for example, by compressing or molding the active ingredient, optionally containing one or more additional components. Compressed tablets may be prepared by compressing, in a suitable device, the active ingredient in a free-flowing form such as a powder or granular preparation, optionally mixed with one or more of a binder, a lubricant, a glidant, an excipient, a surface active agent, and a dispersing agent. Molded tablets may be made by molding, in a suitable device, a mixture of the active ingredient, a pharmaceutically acceptable carrier, and at least sufficient liquid to moisten the mixture.

Tablets may be non-coated or they may be coated using methods known in the art or methods to be developed. Coated tablets may be formulated for delayed disintegration in the gastrointestinal tract of a subject, for example, by use of an enteric coating, thereby providing sustained release and absorption of the active ingredient. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional components including, for example, an inert solid diluent. Soft gelatin capsules comprising the active ingredient may be made using a physiologically degradable composition, such as gelatin. Such soft capsules comprise the active ingredient, which may be mixed with water or an oil medium.

Liquid formulations of a pharmaceutical composition of the invention which are suitable for administration may be prepared, packaged, and sold either in liquid form or in the form of a dry product intended for reconstitution with water or another suitable vehicle prior to use.

Liquid suspensions, in which the active ingredient is dispersed in an aqueous or oily vehicle, and liquid solutions, in which the active ingredient is dissolved in an aqueous or oily vehicle, may be prepared using conventional methods or methods to be developed. Liquid suspension of the active ingredient may be in an aqueous or oily vehicle and may further include one or more additional components such as, for example, suspending agents, dispersing or wetting agents, emulsifying agents, demulcents, preservatives, buffers, salts, flavorings, coloring agents, and sweetening agents. Oily suspensions may further comprise a thickening agent. Liquid solutions of the active ingredient may be in an aqueous or oily vehicle and may further include one or more additional components such as, for example, preservatives, buffers, salts, flavorings, coloring agents, and sweetening agents.

Powdered and granular formulations according to the invention may be prepared using known methods or methods to be developed. Such formulations may be administered directly to a subject, or used, for example, to form tablets, to fill capsules, or to prepare an aqueous or oily suspension or solution by addition of an aqueous or oily vehicle thereto. Powdered or granular formulations may further comprise one or more of a dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. Such compositions may further comprise one or more emulsifying agents. These emulsions may also contain additional components including, for example, sweetening or flavoring agents.

Suitable compositions can comprise from about 100 mg to about 3000 mg of niacinate per unit dose, and may contain up to about 5 gm of IHN.

Inositol hexaniacinates are generally prepared from the desired inositol stereoisomer by reaction with six equivalents nicotinyl chloride hydrochloride in refluxing anhydrous pyridine, as illustrated in Scheme 3.

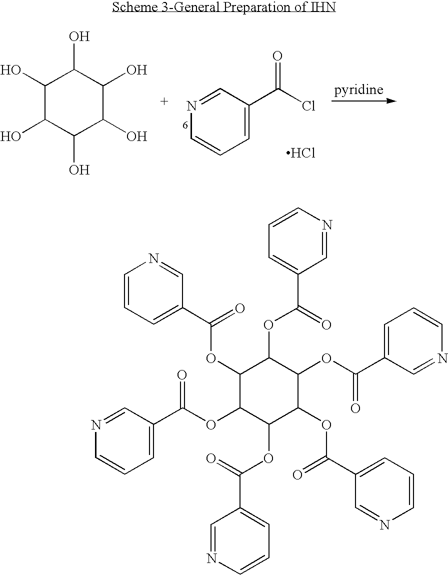

In most cases instances, excess nicotinyl chloride hydrochloride is added. In the case of the scyllo isomer, inositol niacinates that were not completely esterified, i.e. tetra- and penta-niacinates, were reacted with additional niacin or nictotinyl chloride hydrochloride to provide the hexa-ester.

Scyllo-inositol is not widely available from commercial sources. Accordingly, this isomer was synthesized. Several synthetic approaches to scyllo-inositol are known. For example, U.S. Published Patent Application No. 2006/0240534 is directed to a process for producing scyllo-inositol using a microorganism to convert myo-inositol to scyllo-inositol. Scyllo-inositol was indicated as a therapeutic agent for treating Alzheimer disease.

Methods of producing scyllo-inositol by means of a chemical synthetic procedure include: (i) reducing hexahydroxybenzene with Raney nickel; (ii) reducing scyllo-inosose obtained from a glucofuranose derivative through a reaction involving five steps; (iii) a four step reaction starting from cis-trioxa-tris-homobenzene; and (iv) oxidizing myo-inositol with a platinum catalyst to thereby obtain scyllo-inosose, and subjecting the scyllo-inosose to esterification followed by reduction and hydrolysis.

It is also known to convert myo-inositol into scyllo-inositol or scyllo-inosose using a bacterium belonging to the genus Agrobacterium. However, this method is not an industrially viable method because of low yield of scyllo-inositol and generation of other side products.

The enzyme which oxidizes myo-inositol into scyllo-inosose (myo-inositol 2-dehydrogenase) is found in a number of organisms such as animals, algae, yeasts, and bacteria. Examples of a typical microorganism having the enzyme include Aerobacter aerogeties, bacteria belonging to the genus Bacillus and bacteria belonging to the genus Pseudomonas.

Another method of producing scyllo-inositol is by the chemical reduction of scyllo-inosose produced by microbial oxidation. The substance obtained by the chemical reduction of scyllo-inosose is a mixture of scyllo-inositol and myo-inositol, and has to be desalted and purified, followed by separation of scyllo-inositol from the concentrated solution by crystallization. Such methods have required many operations and thus there has been room for improvement with respect to the yield of scyllo-inositol.

When scyllo-inosose is reduced using NaBH₄ in a solution, the resultant reaction product solution contains myo-inositol, scyllo-inositol, and a scyllo-inosito/boric acid complex. The complex is removed as a precipitate; dissolved in diluted sulfuric acid; and methanol is added to form an azeotrope with boric acid. The boric acid is removed and the remaining solution is desalted using an ion exchange resin.

Based on the chemical scheme set forth in the literature for producing scyllo-inositol from myo-inositol, the myo-form being readily available, (“Improved Synthesis of Scyllo-inositol and its Orthoformate from Myo-inositol”, Carbohydrate Research 338: 999-1001 (2003)), high purity scyllo-inositol was formed using the reaction sequence shown in Scheme 4.

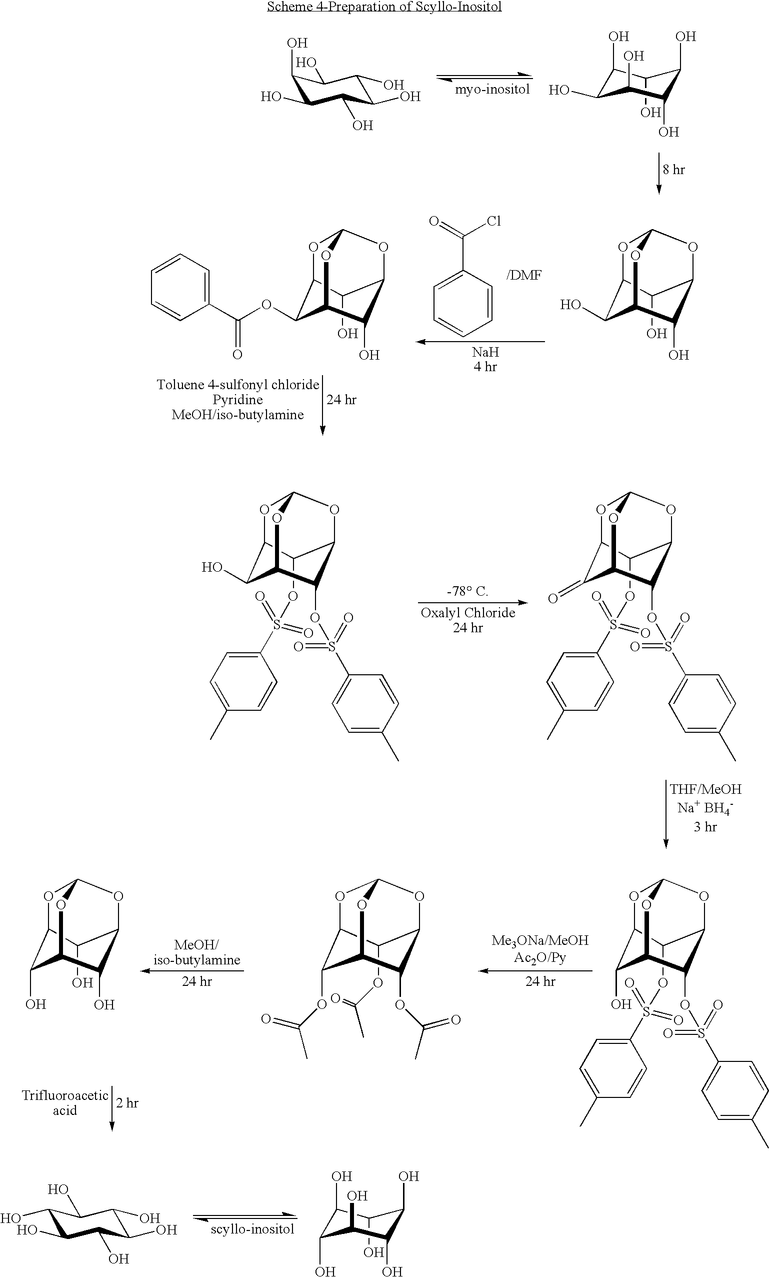

### Basically,

1. Myo-inositol ortho-formate was first produced from myo-inositol.
2. The diol was then protection using toluenesulfonyl chloride (tosylated) and was oxidized using oxalyl chloride at −78° C. The use of the extremely low temperature in the oxidation step ensures stability of the compound and avoids destruction of formate moeity.
3. Sodium Borohydride reduction results in —OH production with the scyllo-configuration (alternating three up, three down).
4. Deprotection of the tosylate part was accomplished using acetate (acetic anhydride), followed by mild hydrolysis with isobutylamine, to produce scyllo-orthoformate.
5. Trifluoroacetic acid (TFA) was then used to hydrolyze the orthoformate to obtain scyllo-inositol.

As an alternative, an acetonide-like group can be used instead of tosylate to protect the formate inositol from destruction during the subsequent reactions

While a preferred reaction scheme to convert myo-inositol to scyllo-inositol is shown, one skilled in the art will recognize that other procedures and different starting compositions can be used to obtain the scyllo-isomer.

Scyllo-IHN cab also be prepared from the reaction of scyllo-inositol and nicotinoyl chloride hydrochloride under reflux in anhydrous pyridine. (See Scheme 3) Owing to the high cost of scyllo-inositol, the synthetic route of scyllo-IHN was first explored using myo-inositol as a template. Once the conditions for the preparation of myo-IHN were optimized attempts to produce scyllo-IHN were made. It was at this time that the striking differences in solubility between myo-IHN and scyllo-IHN were observed as scyllo-IHN and the intermediates to scyllo-inositol hexanicotinate (tetra and penta) were poorly soluble and crystallized from the solution during the synthetic procedure. However, 90% purity of scyllo-IHN was eventually obtained after resubmitting synthetic intermediates to react with niacin. The identity and purity of recovered scyllo-IHN were obtained from LC-MS.

As described below, based on a structural analysis of the various inositol isomers and inositol hexaniacinates formed from the different inositol isomers, it was believed that myo-IHN may not the most beneficial form of IHN for delivering niacin to the body. Based on a simplistic structural analysis, scyllo-IHN was predicted to have preferred properties as compared to myo-IHN due to its significantly reduced steric hindrance. However, experimental results, argue against this conclusion and it was unexpectedly found that allo-IHN has a physical chemical property profile that makes it better suited for physiological release of niacin than myo-IHN. Other IHN isomers can similarly have properties that make them favorable to myo-IHN as therapeutic agents.

### Structural Analyses

A theoretical-mathematical analysis was used as a tool to predict differences in physical chemical properties of inositol and inositol hexaniacinate stereoisomers as a function of conformational geometry and molecular stereochemistry. Calculations of the following parameters and properties were performed:
Heat of formation as a characteristic of molecule stability,
Dipole moment as indication of polarity, and
Steric energy as a representation of relative conformational stability.

Calculations were performed using the semi-empirical quantum-mechanical method PM3. “MOPAC 2000” J. J. P. Stewart, Fujitsu Limited, Tokyo, Japan (1999). The KEYWORDS used for geometry optimization (set forth in the Mopac 2000 manual) were: LET DDMIN=0.0001 EF H20. Application of H₂O settings allows simulation of the effect of water as a medium.

Heat of Formation and Dipole moment were obtained from the minimum energy conformer. Pure Steric Energy was calculated from the PM3 minimum energy conformers geometry using a molecular mechanic approach (MM2: Allinger, N. L., J. Amer. Chem. Soc., 99, 8127 (1977). Burkert, U. and N. L. Allinger, Molecular Mechanics, American Chemical Society: Washington, D.C., 1982.; MM3: Allinger, N. L., Y. H. Yuh, and J. H. Lii, J. Amer. Chem. Soc., 111, 8551 (1989). Lii, J. H. and N. L. Allinger, J. Amer. Chem. Soc., 111, 8566 (1989). Lii, J. H. and N. L. Allinger, J. Amer. Chem. Soc., 111, 8576 (1989).)

The Chem3D graphic interface was used to build the molecular models and to visually evaluate possible geometric configurations.

Confonmational freedom of four different inositol isomers, scyllo-, myo-, cis- and allo-, were explored in order to find the most stable geometries. The most stable conformers from each of the evaluated inositol isomers, were then used to build inositol-hexaniacinate molecules and these were analyzed in order to determine any relevant differences from the then modynamic and structural point of view.

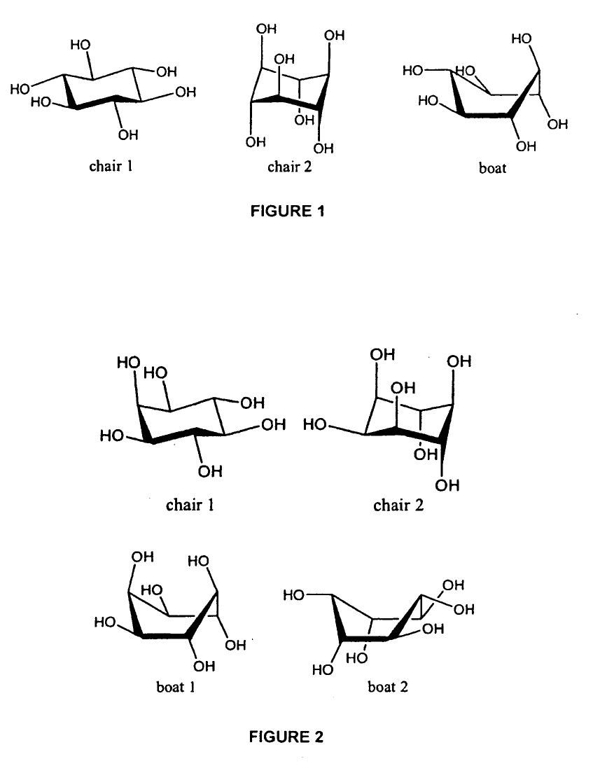

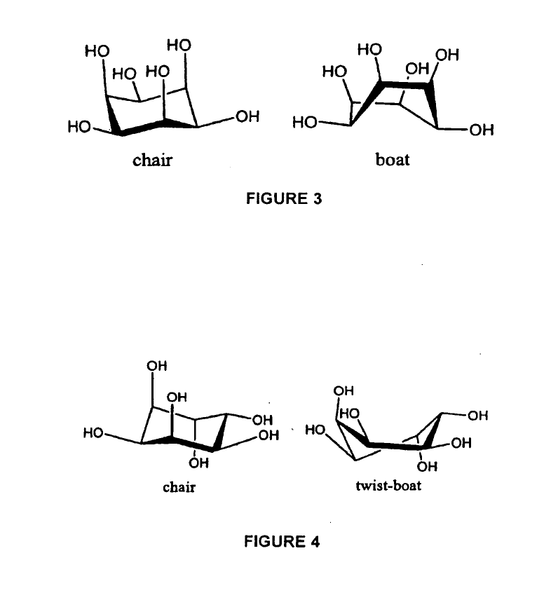

FIGS. 1-4 show the main conformations of the scyllo-, myo-, cis- and allo-inositol isomers, respectively. Table 1 lists the heat of formation (PM3), dipole moment (μ) and steric energy (MM2 and MM3) determined for these four isomers of inositol.

The Heat of Formation, ΔH_{f0}, is the heat evolved from the synthesis reaction of one mole of the substance from the standard state of its constituent elements. It is an indication of the thermodynamic stability of a molecular system. The heat of formation of a substance is a measure of how much internal energy it has, or its ability to produce heat when reacted. Substances with a positive heat of formation are less stable energetically than the elements from which they are formed. Heat of formation differences between isomers and or conformers allow for an estimate of which specific molecular geometry will be thermodynamically favored, and hence, more abundant.

Steric Energy, derived from a molecular mechanics approach, is a measure of the molecular strain. Steric energy is the summation of individual contributions, namely: stretch energy, bend energy, torsion energy, and nonbonded interactions (Van der Valls, dipole-dipole, electrostatic, etc.). The set of equations required to describe the behavior of a specific arrangement of atoms and bonds, is called a force-field. Many different kinds of force fields have been developed over the years (MM2, MM3, AMBER, etc). Some include additional energy terms that describe other kinds of deformations. The object of molecular mechanics is to predict the energy associated with a given conformation of a molecule. Table 1 lists the alternative calculations of steric energy from different force fields (MM2 and MM3). Calculated molecular mechanics energies have no meaning as absolute quantities; only differences in energy between two or more conformations have meaning and provide the opportunity to compare energies of different conformations of the same molecule as well as energies of different stereoisomers, such as diasteroisomers. The energies of molecules with different numbers of atoms cannot be compared nor can one compare energies calculated using different force fields. Molecules with multiple free rotating bonds generate a complicated distribution of energy vs. geometry; therefore, multiple energy minima can be found.

Dipole Moment is produced by the inhomogeneous electron charge distribution in a molecular structure. Such differences in the electron-density distribution create a dipole vector. The dipole vector is significant when considering the solubility behavior of a given molecule in a given solvent. From a simplistic point of view, molecules with a higher positive dipole moment value (negative value for dipole moment does not have scientific value) will dissolve better in polar solvents; molecules with no dipole moment or a dipole moment close to zero will be solubilized better in non-polar solvents. However, the dipole moment is not the only parameter relevant to solubility. The ability to generate hydrogen bonding, polar to non-polar surface ratio, etc., are also important descriptive parameters related to solubility. Irrespective thereof, the net dipole moment is a generally accepted approach to understand how a molecule would behave in a specific solvent medium in comparison to other similar molecules. Comparison of the molecule properties based on Dipole Moment values is applicable in absolute values for homologues, and isomers, as is done herein. Other physical chemical properties of different classes of compounds can significantly affect the ability to make predictions based on dipole moment. Because properties of similarly structured compounds are being analyzed, prediction of solubility of the compounds in polar solvents, such as, a bio fluid, is viable.

The three-dimensional shape of the molecule is also very important when considering the interaction with an enzyme. The interaction with an active-site, requires the molecule to fit into the specific three-dimensional distribution of the receptor. The affinity can be altered by factors such as steric hindrance. In regard to the particular isomers under investigation, it is difficult to judge how effective a molecule will interact with an enzyme without an understanding of the three dimensional fitting between the two entities. The simpler criteria applicable in this instance is that the less the steric hindrance in the vicinity of the target functional group, the greater possibility to interact chemically with other entities

TABLE 1

| | | DIPOLE | | |
|---|---|---|---|---|
| | HEAT | MO- | | |
| | OF FORMATION | MENT | STERIC ENERGY | |
| Conformers | PM3 Hf (Kcal/mol) | μ (Debye) | MM2 (kcal/mol) | MM3 (Kcal/mol) |
| Scyllo-inositol | | | | |
| 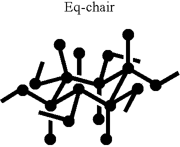 | −273.51 | 0.015 | −0.772 | 0.353 |
| 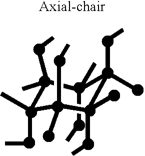 | −270.97 | 1.714 | −2.151 | 16.239 |
| 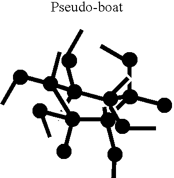 | −269.73 | 1.979 (2.198)* | 2.918 | 19.330 |
| Myo-inositol | | | | |
| 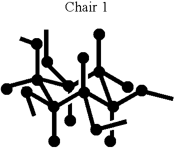 | −273.81 | 0.283 (2.371)* | −0.405 | 11.282 |
| 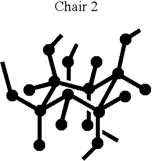 | −271.28 | 4.064 | −2.529 | 14.357 |
| 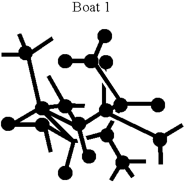 | −269.88 | 1.535 | 3.021 | 22.358 |
| 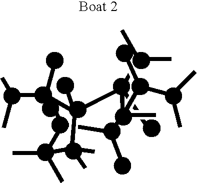 | −270.61 | 1.894 | 0.533 | 19.63 |
| cis-inositol | | | | |
| 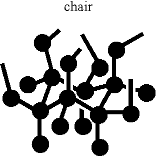 | −275.41 | 2.041 | −3.851 | 6.763 |
| 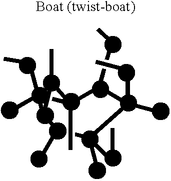 | −269.45 | 1.947 | −3.548 | 19.250 |
| allo-inositol | | | | |
| 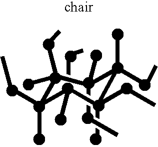 | −271.87 | 3.743 | −1.429 | 7.559 |
| 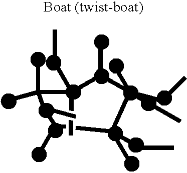 | −270.57 | 3.985 (1.303)* | 0.942 | 15.570 |

Generally, the chair-like conformations of the inositol isomers were the most thermodynamically stable forms as estimated by minimization of heat of formation or the molecular mechanic approach. Theoretical calculations support that observation; in all cases, chair conformation allows the best steric accommodation of the hydroxyl groups, producing less hindrance, and, correspondingly, less steric energy. The ability to form intra-molecular hydrogen-bonding between the hydroxyl groups produces an additional source of molecular geometry stabilization. It should be noted that a full conformational-study would include a greater conformational range; and, possibly more accurate estimation of the effect of the solvent medium, in this instance water, on thermodynamic properties. The lack of ability to estimate the effect of the medium using different mathematical approaches might introduce aberrations in the conclusions. However, the limited theoretical analysis set forth herein supports the conclusion that the chair-conformation is the most stable configuration and, apparently, scyllo-inositol is the most thermodynamic stable isomer from the structural point of view. Therefore, the chair conformations were selected to be the starting point to build the hexaniacinate (IHN) molecules.

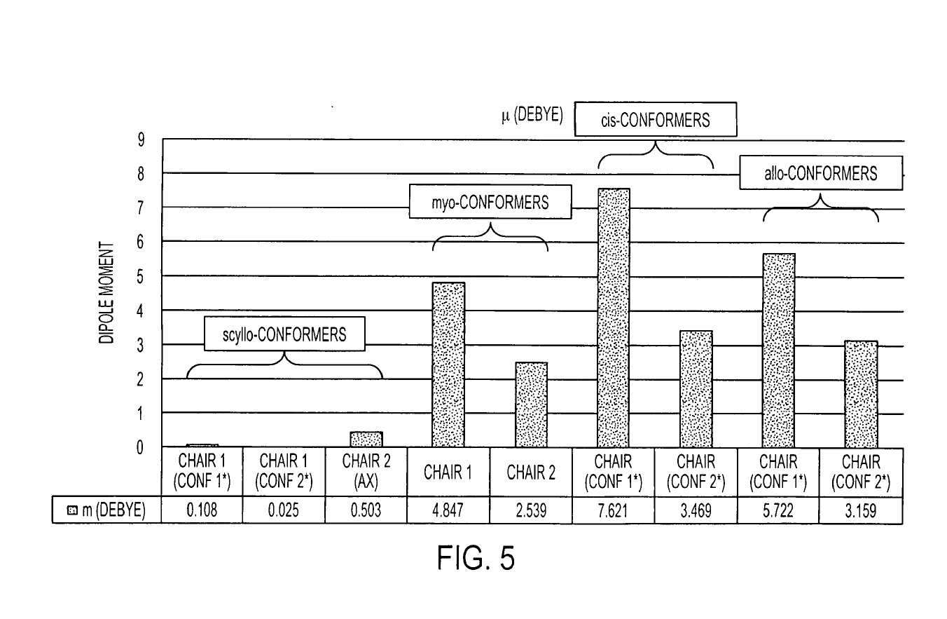

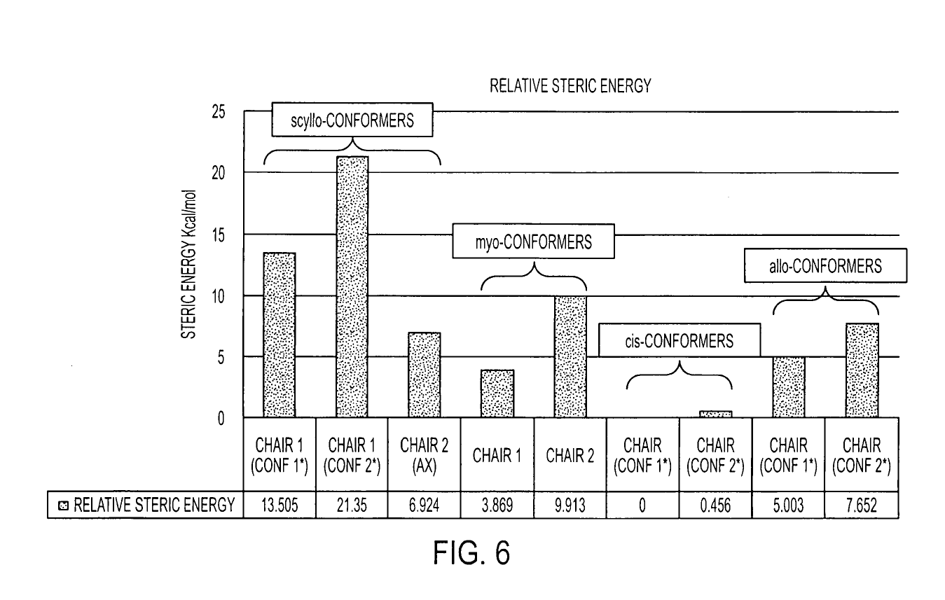

Based on the physical characteristics calculated for the various inositol isomers it was determined that the scyllo-, cis- and allo-isomers appeared to be the best candidates for production of isomeric inositol hexaniacinate compounds having superior physiological properties and/or dissolution properties once delivered to patients for treatment of, or prevention of, diseases that appear to be amenable to niacin treatment, while at the same time reducing or eliminating the side effects from delivery of niacin in its various release forms. Table 2 lists the calculated heats of formation, dipole moments and steric energies for the hexaniacinate esters of scyllo-, cis-, myo-, and allo-inositol chair conformations. The calculated dipole moments and relative steric energies of the various compounds are shown graphically in FIGS. 5 and 6 respectively.

TABLE 2

| | | | |
|---|---|---|---|
| | PM3 Hf | | |
| Conformers¹ | (Kcal/mol) (Δ)² | μ (Debye) | MM2 (kcal/mol)³ |

scyllo-inositol hexanicotinate

| | | | |
|---|---|---|---|
| Chair 1 (conf 1*) | −241.96 (0.0) | 0.108 | 25.905 (13.5) |
| Chair 1 (conf 2*) | −236.25 (5.7) | 0.025 | 33.750 (21.4) |
| Chair 2 (ax) | −238.71 (3.3) | 0.503 | 19.324 (6.9) |

myo-inositol hexanicotinate

| | | | |
|---|---|---|---|
| Chair 1 | −241.72 (0.2) | 4.847 | 16.269 (3.9) |
| Chair 2 | −236.68 (5.3) | 2.539 | 22.313 (9.9) |

cis-inositol hexanicotinate

| | | | |
|---|---|---|---|
| Chair (conf 1*) | −234.64 (7.3) | 7.621 | 12.400 (0.0) |
| Chair (conf 2*) | −234.25 (7.7) | 3.469 | 12.856 (0.5) |

allo-inositol hexanicotinate

| | | | |
|---|---|---|---|
| Chair (conf 1*) | −240.63 (1.3) | 5.722 | 17.403 (5.7) |
| Chair (conf 2*) | −234.52 (7.4) | 3.159 | 20.052 (3.2) |

¹Figures with structures are further presented in this document
²Difference between observed value and smallest obtained heat of formation value in examined species
³Optimized starting from the PM3 generated structure
*Different spatial configuration for the ester group

The published literature (“MM3 (92) Analysis of Inositol Ring Puckering”, Australian J. Chem. 49(3):327-335 (1996)) indicates that scyllo-inositol is the most stable of the inositol isomers. Calculations also show that the scyllo-inositol is the least sterically hindered of the inositol isomers and is 50% less sterically hindered than the myo-inositol isomer. Comparison of the 3-D form of myo-IHN in its lowest energy state conformation with the 3-D form of scyllo-IHN in its lowest energy state conformation clearly shows this reduced steric hinderance. Irrespective of the fact that scyllo-inositol is more stable, it was initially believed, because of the significantly reduced steric hindrance, that the first niacin moiety attached to scyllo-IHN would hydrolyze faster when exposed to hydrolytic enzymes, i.e., in the presence of plasma esterases, when compared to myo-IHN. Since the speed with which the first niacin moiety is hydrolyzed is expected to be the rate limiting step in the hydrolysis of the niacin moieties on IHN, it was postulated that scyllo-IHN would result in a faster and larger increase of free niacin in human plasma than the myo-IHN and a faster onset of action and a lessened release profile, which in turn would result in better lipid lowering and cardiovascular benefits as well as circulatory benefits for conditions such as intermittent claudication and Raynaud's disease.

By performing an MM2 calculation, the steric energy associated with different molecular structures was determined. These values are illustrated graphically in FIG. 6. It was concluded that scyllo-IHN has less steric hindrance than myo-IHN and as a result it was believed that the first moiety of niacin would be released by scyllo-IHN faster than from myo-IHN. Initial results indicated that this premise is false in a physiological medium and that the expected superiority of scyllo-IHN was not observed. Based on calculations and observed data, it is now believed that the polarity of the various isomers, shown in FIG. 5, may be a more important factor than steric hindrance. Specifically, scyllo-IHN is more non-polar and does not dissolve in the relevant body fluids, and, as a result, it is not decomposed or metabolized in the body and no or little niacin is released. In contrast, allo-IHN is the most polar form and therefore more readily dissolves. Dissolution appears to be an important step in the release of niacin from IHN isomers and, as a result, allo-IHN releases niacin more efficiently than myo-IHN or scyllo-LIN.

Data show that different IHN stereisomers have unexpectedly different physical chemical properties and can release niacin at different rates, which are dependent on the conditions. For example, in SGF at pH 1.1, there is little difference in the hydrolytic rates between allo-IHN and myo-IHN. Although allo-IHN dissolves faster than myo-IHN, the rate of release of niacin is similar from both isomers. However, in pH 7.4 phosphate buffer with or without esterase, the solubility and subsequent hydrolysis of myo-IHN are lower than that of allo-IHN. This is apparently related to the improved solubility of allo-IHN in pH 7.4 aqueous media in comparison to myo-IHN. It is also notable that the presence of esterase enhances the release of niacin from allo-IHN but not myo-IHN.

The difference in the solubility of myo-IHN and allo-IHN in the above test media is consistent with the calculated dielectric constants. Allo-IHN conformations have greater calculated dielectric constants than myo-IHN (allo isomer μ>3 Debye, myo isomer μ>2.5 Debye). As anticipated allo-IHN possesses better solubility under both acidic (SGF) and neutral conditions (pH 7.4 phosphate buffer) than scyllo- and myo-IHN.

The nonpolar nature of scyllo-IHN and its resulting poor solubility in SGF is likely the determining factor in its hydrolysis. Myo-IHN has much better solubility in SGF and therefore hydrolizes more readily than scyllo-IHN. The difference in the solubility between myo-IHN and scyllo-IHN in SGF is supported by the calculated dielectric constants set forth above. Conformations of scyllo-IHN have calculated dielectric constants close to zero when compared to myo-IHN (μ>2.5 Debye). The striking difference in the solubility of myo-IHN and scyllo-IHN results in the different hydrolytic rates observed in the test media. On the other hand allo-IHN has a calculated dielectric constant greater than about 3 Debye.

Based on calculations and experimental data, it is expected that allo-IHN will be more soluble than myo-IHN in the intestines and will provide a controllable and more rapid release of niacin into the blood stream. Allo-IHN can therefore provide a more effective treatment and a higher effective dosage of niacin with a reduced requirement for ingested IHN to obtain the results previously experienced using niacin therapy in treating various medical conditions. Because it has a greater polarity than myo-IHN, cis-IHN is also expected to be superior to myo-IHN. At least allo-IHN and cis-IHN are therefore usable in all conditions where niacin delivery has been found to be effective and its use will result in reduced side effects because niacin is delivered more readily than from myo-IHN. Other IHN isomers may also have increased solubility and be superior to myo-IHN. Other IHN isomers can have properties that are more suitable for particular applications. For example, the resistance of solubilization demonstrated by scyllo-IHN may find uses in particular applications.

In addition, allo-IHN and isomers with similar physical chemical and physiological properties are expected to release niacin in a controlled and more effective manner, and is therefore likely to be effective in situations where niacin has been indicated as ineffective or contra-indicated because of the effect of niacin on the liver.

Problems associated with the administration of niacin can be alleviated by delivering niacin by the administration of particular IHN stereoisomers such as allo-IHN because IHN may pass through the liver and deliver niacin directly to the bloodstream. Further, the apparent lack of benefit of niacin delivery in some instances or the inconsistent results using niacin can now be eliminated by the ability to control the release of niacin from particular IHN stereoisomers such as allo-IHN. In addition, combination therapy with statins, counter-indicated in the past due to liver problems, may now be viable and allow statin dosages to be reduced.

Particular IHN stereoisomers such as allo-IHN and cis-IHN can be unexpectedly superior models for prodrug development. The combination of higher dipole moment and lower steric energy, due to the specific spatial distribution of nicotinoyl groups, suggests that these particular isomers would be relatively more soluble in polar and medium polar solvents, easier to synthesize since there is less global steric hindrance in these structures; and under chemical and/or enzymatic hydrolysis, the release of niacin molecules in bio-fluids is easier because of the better accessibility to the ester groups. Based on the same reasoning, any other IHN stereoisomers with dipole moments greater than myo-IHN would be more soluble and more readily hydrolyzed to release niacin than myo-IHN.

### EXAMPLES

### Example 1

### Synthesis of Allo-Inositol Hexaniacinate

Allo-IHN was prepared by reacting allo-inositol with six equivalents nicotinoyl chloride hydrochloride under reflux in anhydrous pyridine. Allo-IHN was produced within 5 hours with 95% purity. One more equivalent of nicotinoyl chloride hydrochloride (˜100 mg) was then added and the reaction continued overnight. The reaction was quenched by addition of DI water and the excess amount of nicotinoyl chloride was converted into niacin. The product was then purified using a C18 cartridge. Niacin, pyridine and water soluble contaminants were removed from the C18 column by washing with DI water. The allo-IHN was then eluted from the column with acetonitrile, the acetonitrile fractions were collected and their contents were verified by HPLC and combined. After evaporating the solvent, allo-IHN was obtained with 98.5% purity. The purity and identity of allo-IHN was confirmed by HPLC and LC-MS (Model: Q-T of Micro, serial No. YB314).

### Example 2

### Synthesis of Scyllo-Inositol Hexaniacinate

Scyllo-inositol was prepared from myo-inositol by a method based on the chemical scheme set forth in the literature. “Improved Synthesis of Scyllo-inositol and its Orthoformate from Myo-inositol”, Carbohydrate Research, 338: 999-1001 (2003). In summary, myo-inositol ortho-formate was first produced from myo-inositol and the equatorial hydroxyl esterified with benzoyl chloride. The diol was then protection using toluenesulfonyl chloride (tosylated), the benzoyl group removed and the hydroxyl oxidized using oxalyl chloride at −78° C. The use of the extremely low temperature in the oxidation step ensures stability of the compound and avoids destruction of formate moeity. Sodium borohydride reduction results in —OH production with the scyllo-configuration (alternating three up, three down). The tosylate was removed with acetic anhydride, followed by mild hydrolysis with isobutylamine, to produce scyllo-orthoformate. Trifluoroacetic acid (TFA) was then used to hydrolyze the orthoformate to obtain scyllo-inositol.

Scyllo-IHN was prepared from the reaction of scyllo-inositol and nicotinoyl chloride hydrochloride under reflux in anhydrous pyridine. The reaction process was monitored by TLC and LC-MS. It was observed that scyllo-IHN and the tetra- and penta-esters were poorly soluble and crystallized from the solution during the synthetic procedure. However, 90% purity of scyllo-IHN was obtained by resubmitting the tetra- and penta-esters and subjecting them to further reaction with niacin. The identity and purity of recovered scyllo-IHN were verified by LC-MS.

### Example 3

### Dissolution and Hydrolysis in Simulated Gastric Fluid

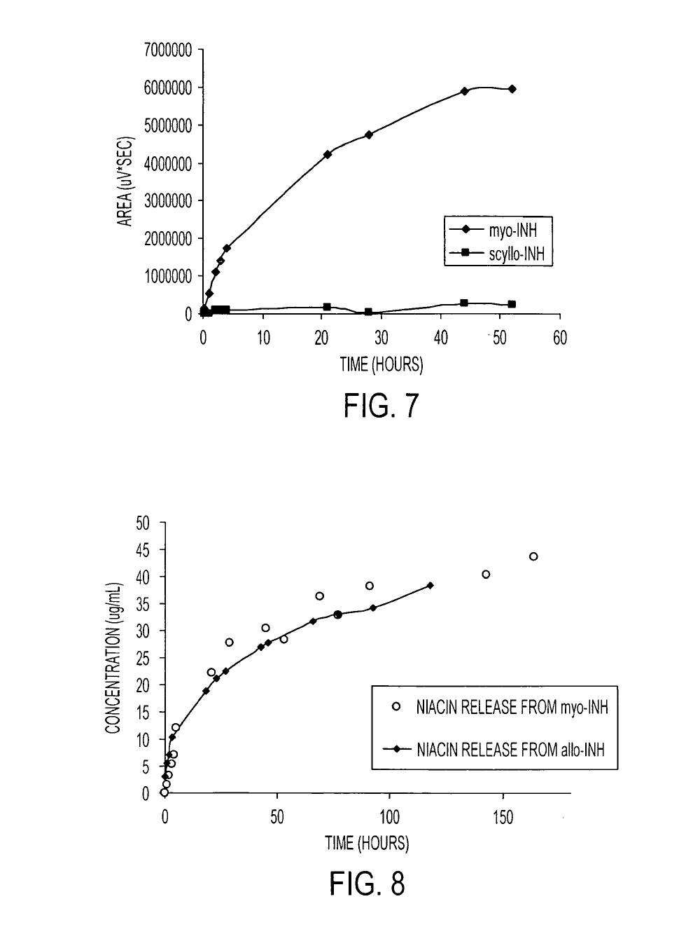

A comparative study of the hydrolysis of myo-IHN and scyllo-IHN in simulated gastric fluid (SGF) test solutions was conducted. Reaction mixtures were prepared by dispersing 25 mg of myo-IHN or scyllo-IHN in 25 mL of SGF (pH 1.1). The hydrolysis was performed in a 37±1° C. thermostatic water bath with a shaking rate at 97±2 rpm. At various time intervals, 100 μl aliquots were taken from the reaction mixture and diluted with 1.5 mL 80/20 acetonitrile/formic acid which were used to quench the hydrolysis reaction. The solubility of scyllo-1-HN was found to be very poor in the SGF test solution and solid crystals still remained floating on the liquid surface after 53 hours. Myo-IHN, however, dissolved completely after 6 hours. FIG. 7 shows a comparative presentation of the release of niacin from myo-IHN and scyllo-IHN in SGF up to 53 hours. The poor solubility observed in scyllo-IHN is apparently the result of its symmetrical nature as well as the nonpolar nature of scyllo-IHN. The poor dissolution of scyllo-IHN in SGF limits the ability of this compound to hydrolyze.

After 53 hours, 2 ml of concentrated HCl was injected into the reactor containing scyllo-IHN, while the reaction of myo-IHN remained undisturbed. It was found that by increasing the acidity of the reaction medium by adding 0.2 HCl, the scyllo-IHN can be dissolved and the hydrolysis reaction commences to produce niacin. However, these conditions are significantly more acidic than would be expected in the human stomach. This experiment supports the importance of dissolution in the hydrolysis process and the conclusion that the more soluble allo-IHN and cis-IHN are in fact preferred over the scyllo-IHN and myo-IHN under these conditions.

The hydrolysis of allo-IHN was performed in simulated gastric fluid (SGF). The test solution was prepared according to USP standard procedure (USP29-NF24 S2) without the addition of pepsin (prior test showed the addition of pepsin had no effect on the dissolution/hydrolysis of IHN). The pH of the SGF solution was 1.1 at 22° C. The hydrolysis mixture was prepared by dispersing 20 mg allo-IHN in 200 ml of SGF test solution. The reactor was placed in a thermostatic water bath at 37±1° C. with a shaking rate at 42±1 rpm. At various reaction times, 1 mL aliquots were taken from the reaction mixture and analyzed for niacin by HPLC.

Dissolution and hydrolysis of allo-IHN began immediately after addition to the reaction medium. Ideally the hydrolysis would proceed until all the nicotinoyl substituents are cleaved from the allo-Inositol. The theoretical concentration of niacin at 100% release was expected to be 91 μg/mL (calculated from the concentration of allo-IHN in 20 mg/200 mL in SGF). After allo-IHN hydrolyzed in SGF for 118 hours, 38.5 μg/mL of niacin (˜42% of theoretical niacin content) was released. The appearance of the degradation intermediates of allo-IHN were also monitored. At a later stage of hydrolysis (>100 hours) the release of niacin slowed due to a much slower hydrolytic kinetics involved in the cleavage of niacin from tetra-, tri-, and di-substituted inositol. Similar degradation kinetics were also observed for myo-IHN. More specifically in both instances, once the pentaester was formed, its hydrolysis occurred relatively quickly; however, the tetra-, tri-, and di-substituted isomers showed a slower rate of hydrolysis.

FIG. 8 shows the release of niacin from both allo-IHN and myo-IHN in SGF for up to 150 hours to be about the same. Perhaps due to the similar dissolution properties of both isomers in SGF, there is little difference in the hydrolytic rates for allo- and myo-IHN in SGF. It is therefore postulated that the dissolution of the Inositol Hexaniacinate is an important factor in its hydrolytic rate in SGF.

### Example 4

### Dissolution and Hydrolysis in Phosphate Buffer

### Example 4A

### SIF with Pancreatin at pH 6.7

Simulated intestinal fluid (SIF) with pancreatin was prepared according to the USP standard procedure (USP29-NF24 S2) with a pH of 6.7 at 24° C. It was found that myo-IHN was poorly soluble in SIF and there is very little release of niacin up to 40 hours.

### Example 4B

### SIF without Pancreatin at pH 7.4

A pH 7.4 phosphate buffer solution was also prepared from a SIF test solution in the absence of pancreatin. Myo-IHN was essentially insoluble in pH 7.4 phosphate buffer. In a parallel experiment, 20 mg allo-IHN was added to 200 mL of the pH 7.4 phosphate buffer. The release of niacin and the appearance of the degradation intermediates were monitored up to about 74 hours. Allo-IHN has a much better solubility than myo-IHN under the same conditions. The hydrolytic release of niacin was again observed to depend largely on the improved solubility of allo-IHN. The concentration of niacin reached a maximum of 30.34 μg/mL (˜33% of theoretical release) at about 25 hours with a decrease in the concentration of niacin thereafter.

### Example 4A

### SIF with Esterase at pH 7.4

The hydrolyses of allo-IHN and myo-IHN in pH 7.4 phosphate buffer with esterase were also compared. Esterase is an enzyme found in animal liver which catalyzes the hydrolysis of esters. The esterase reaches maximum activity in pH 8.0 borate buffer at 25° C. Simulated intestinal fluid without pancreatin (SIF, 1 L) was prepared according to USP standard procedure (USP29-NF24 S2). The pH of the SIF solution was then adjusted to 7.4. Esterase (6.0 mg) was then added to 200 mL of the pH 7.4 phosphate buffer. Instead of adding the materials directly as a solid, 20 mg of allo-IHN and 20 mg myo-IHN were separately suspended in 2 mL 0.1N HCl. After sonication for about 1 minute these suspensions were carefully transferred to the hydrolysis medium. As each suspension was added to the pH 7.4 phosphate buffer, a milky white precipitated immediately appeared. The reactors were kept in a thermostatic water bath at 37±1° C. with a shaking rate at 42±1 rpm. At various reaction times ˜2 mL aliquots were taken from the hydrolysis solutions and filtered through 0.45 μm filters in order to remove any undissolved materials. The samples for HPLC analysis were prepared by addition of 20 μL 6N HCl to 1 mL filtrate.

Within one hour, niacin could be detected in both reaction samples. The area response of the niacin peak from the allo-IHN sample was significantly larger than the one from the myo-IHN sample. When the myo-IHN and allo-IHN degradation products in pH 7.4 phosphate buffer with esterase were observed for 16 hours, it was notable that only the degradation intermediates of allo-IHN (penta-, tetra-, tri-, and di-niacinates of inositol) were observed. The absence of degradation intermediates in the myo-IHN sample appears to be due to the fact that only a small fraction of myo-IHN dissolved in the suspension transferred to the pH 7.4 phosphate buffer. On the other hand, the myo-IHN that did dissolve was hydrolyzed and consumed. The dissolution of myo-IHN, however, was considerably slower than the hydrolysis rate. Once there was no supply of myo-IHN in the solution, the hydrolysis ceased. On the other hand, the dissolution of allo-IHN was relatively fast and there was a continuous supply of allo-IHN and the degradation intermediates in the solution.

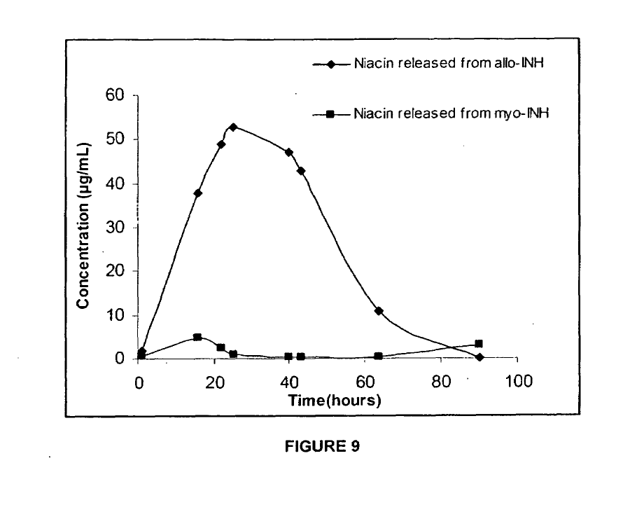

The release of niacin from both myo-IHN and allo-IHN at various reaction times are compared in FIG. 9. The release of niacin from myo-IHN was caused by smaller amount of myo-IHN soluble in 0.1NHCl. The release of niacin ceased after this small fraction of myo-IHN was consumed. Most myo-IHN remained as a solid and did not hydrolyze. This data further supports the importance of dissolution in the hydrolysis process of IHN isomers. The concentration of niacin in the degraded allo-IHN sample reached 52.8 μg/mL (˜58% of theoretical release) at 25 hours.

The data show that the presence of esterase further enhances the release of niacin from allo-IHN. A decrease in the concentration of niacin after 25 hours in the SIF solution was observed and it is believed this is due to the decomposition of niacin under these condition and not due to a decrease in the release of niacin. This is supported by the appearance of new peaks in the HPLC chromatograms.

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

1. A compound comprising an ester formed from an inositol or an inositol derivative and niacin, wherein the inositol or the inositol derivatives comprises a stereoisomer selected from allo-inositol, cis-inositol, epi-inositol, muco-inositol, neo-inositol, scyllo-inositol, D-chiro-inositol and L-chiro-inositol, and pharmaceutically acceptable salts thereof.

2. The compound of claim 1, comprising an inositol hexaniacinate.

3. The compound of claim 1, wherein the stereoisomer of inositol is allo-inositol.

4. The compound of claim 1, wherein the stereoisomer of inositol is cis-inositol.

5. The compound of claim 1, comprising allo-inositol hexaniacinate.

6. The compound of claim 1, comprising cis-inositol hexaniacinate.

7. A composition comprising an ester formed from an inositol or an inositol derivative and niacin and one or more inert ingredients, wherein the inositol or the inositol derivatives comprises a stereoisomer selected from allo-inositol, cis-inositol, epi-inositol, muco-inositol, neo-inositol, scyllo-inositol, D-chiro-inositol and L-chiro-inositol.

8. The composition of claim 7, comprising allo-inositol hexaniacinate.

9. The composition of claim 7, comprising cis-inositol hexaniacinate.

10. The composition of claim 7, further comprising a second pharmaceutically active moiety selected from the group consisting of HMG-CoA reductase inhibitors, statins, fibrates, activators of peroxisome proliferator activated receptors policosanol, phytosterols, tocotrienols, calcium, bile acid sequestrants, and guar gum.

11. The composition of claim 7, further comprising free niacin.

12. A method of treating a disorder treatable with niacin comprising delivering a therapeutically effective amount of a composition comprising an ester formed from an inositol or an inositol derivative and niacin, wherein the inositol or the inositol derivatives comprises a stereoisomer selected from allo-inositol, cis-inositol, epi-inositol, muco-inositol, neo-inositol, scyllo-inositol, D-chiro-inositol and L-chiro-inositol, or pharmaceutically acceptable salts thereof.

13. The method of claim 12, wherein the ester comprises allo-inositol hexaniacinate.

14. The method of claim 12, wherein the ester comprises cis-inositol hexaniacinate.

15. The method of claim 12, wherein the composition further comprises a second pharmaceutically active moiety selected from the group HMG-CoA reductase inhibitors, statins, fibrates, activators of peroxisome proliferator activated receptors policosanol, phytosterols, tocotrienols, calcium, bile acid sequestrants, and guar gum.

16. The method of claim 12, wherein the disorder treatable with niacin is selected from the group consisting of dyslipodemia, hypercholesterolemia, hyperlipidemia, hypertriglyceridemia, hyperlipoproteinemia, hypocholesterolemia hypolipoproteinemia and imbalances of lipids, lipoproteins and/or triglycerides; cardiovascuolar disease; diabetes or inuslin resistance; peripheral vascular diseases including Raynaud's disease, thrombotic risk, intermittent claudication, hypertension, vascular insufficiency and restless leg syndrome and other peripheral artery disease, dysmennorhea, carcinogenesis, anxiety depression, PMS, and treatment of metabolic syndrome due to insulin resistance.

17. The method of claim 12, wherein the disorder treatable with niacin is selected from the group consisting of dyslipodemia, hypercholesterolemia, hyperlipidemia, hypertriglyceridemia.

18. The method of claim 12, wherein the composition further comprises free niacin.

19. A method of providing niacin to an animal for therapeutic purposes comprising administering an ester formed from an inositol or an inositol derivative and niacin, wherein the inositol or the inositol derivatives derivative comprises a stereoisomer selected from allo-inositol, cis-inositol, epi-inositol, muco-inositol, neo-inositol, scyllo-inositol, D-chiro-inositol and L-chiro-inositol, or pharmaceutically acceptable salts thereof.

20. The method of claim 19, wherein the composition is delivered orally.
